(19) United States
(12) Patent Application Publication
(10) Pub. No.: US 2022/0133242 A1
(43) Pub. Date: May 5, 2022

(54) FEATURE GENERATION BASED ON EIGENFUNCTIONS OF THE SCHRÖDINGER OPERATOR

(71) Applicant: KING ABDULLAH UNIVERSITY OF SCIENCE AND TECHNOLOGY, Thuwal (SA)

(72) Inventors: Taous Meriem LALEG, Thuwal (SA); Abderrazak CHAHID, Thuwal (SA)

(21) Appl. No.: 17/430,111

(22) PCT Filed: Feb. 14, 2020
(86) PCT No.: PCT/IB2020/051275

§ 371 Date: Aug. 11, 2021

Related U.S. Application Data
(60) Provisional application No. 62/867,370, filed on Jun. 27, 2019.
(60) Provisional application No. 62/807,515, filed on Feb. 19, 2019.

(51) Int. Cl. A61B 5/00; A61B 5/245

(52) U.S. Cl. CPC A61B 5/7285; A61B 5/245; A61B 5/7278; A61B 5/726

(57) ABSTRACT

A method for generating a feature associated with input data includes receiving the input data; projecting the input data with a set of square functions ψₙₕ² of the Schrödinger operator; selecting the feature to be a number of the negative eigenvalues λₙₕ of the Schrödinger operator; and classifying the input data based on the feature.

### CROSS-REFERENCE TO RELATED APPLICATIONS

This application claims priority to U.S. Provisional Patent Application No. 62/807,515, filed on Feb. 19, 2019, entitled “SIGNAL CHARACTERIZATION USING SQUARED EIGENFUNCTIONS OF THE SCHRODINGER OPERATOR FOR FEATURE GENERATION,” and U.S. Provisional Patent Application No. 62/867,370, filed on Jun. 27, 2019, entitled “FEATURE GENERATION BASED ON EIGENFUNCTIONS OF THE SCHRODINGER OPERATOR,” the disclosures of which are incorporated herein by reference in their entirety.

### BACKGROUND

### Technical Field

Embodiments of the subject matter disclosed herein generally relate to generating a feature that characterizes input data, and more specifically, to extracting discriminative properties of signals that make up the input data and to use these properties to classify the input data.

### Discussion of the Background

With the advance of electronic devices in each science field, the amount of signals generated and collected for analysis has increased tremendously. Generally, those that analyze the collected signals, in any science field, look for a peak in the signal, where the peak usually is significative for characterizing the phenomenon that is under study. Thus, a common task for these projects is detecting the maximum or minimum of a given collected signal. While this task is conventional for a clean signal, if the signal is very noisy or if the recorded data includes many signals, the task becomes more complicated.

In a concrete example, the presence of peaks in biomedical signals often reflects different chemical and/or biological activities in the human body. An example of such signal is the Magnetoencephalography (MEG) signal. The MEG is a functional neuroimaging modality that measures the magnetic activity of the brain. It uses an array of highly sensitive sensors or magnetometers, called superconducting quantum interference devices (SQUIDs). The MEG signal is less distorted by the intervening tissues between the neural source and the SQUIDs comparing to electroencephalogram (EEG) signal. The MEG signals are very useful for the detection and the treatment of various diseases in the brain as the MEG signals help in localizing the region of the brain which produces the abnormal electrical activities which causes the neurological disorder.

Due to the recent introduction of the MEG method in clinical practice, the spike detection using MEG signals is an emerging research field. Different methods were proposed for spikes detection using EEG/MEG signals. However, few methods exists for spike detection using the MEG signal [1], [2], [3]. For example, in [1], an independent component analysis (ICA) method has been proposed with a multi-channel MEG spikes localization method, which decomposes spike-like and background components into separate spatial topographies and associated time series. The detection is performed using a thresholding technique. Another method is the common spatial patterns (CSP) and linear discriminant analysis (LDA) method (CSP-LDA) [2]. The latter is similar to the method in [1] except for the classification stage, where the LDA classifier is used for the detection.

Moreover, an Amplitude Thresholding and Dynamic Time Warping (AT-DTW) approach has been proposed in [3]. This method uses amplitude thresholding to localize abnormal activities, to specify the region in the brain where the abnormalities happen, and to select the affected channels. For the spike detection, the method employs dynamic time warping. The previously cited works reported a maximum sensitivity and specificity of 92.4% and 95.8%, respectively. Most of these methods study the MEG signal in its time domain, and some of them do not take advantage of recent advances in machine learning classifiers due to the complexity of the MEG signal.

Therefore, a pertinent characterization of MEG signals, which allows the definition of discriminative features, is necessary for an effective use of classifiers for spike detection. It is also necessary that the introduced feature vector is of reduced dimension to improve the effectiveness of the classification

However, the existing methods are still not optimal in terms of feature generation and size of the feature. Thus, there is a need for a new method for feature generation that can overcome these limitations.

### SUMMARY

According to an embodiment, there is a method for generating a feature associated with input data. The method includes receiving the input data; projecting the input data with a set of square functions ψₙₕ² of the Schrödinger operator; selecting the feature to be a number of the negative eigenvalues λₙₕ of the Schrödinger operator; and classifying the input data based on the feature.

According to another embodiment, there is a computing device for generating a feature associated with input data. The computing device includes an interface for receiving the input data; and a processor connected to the interface. The processor is configured to project the input data with a set of square functions ψₙₕ² of the Schrödinger operator; select the feature to be a number of the negative eigenvalues λₙₕ of the Schrödinger operator; and classify the input data based on the feature.

According to still another embodiment, there is a non-transitory computer readable medium including computer executable instructions, wherein the instructions, when executed by a processor, implement instructions for generating a feature as in the method discussed above.

### BRIEF DESCRIPTION OF THE DRAWINGS

The accompanying drawings, which are incorporated in and constitute a part of the specification, illustrate one or more embodiments and, together with the description, explain these embodiments. In the drawings:

FIG. 1 is a schematic illustration of a method that applies the Schrödinger operator for reconstructing a signal;

FIG. 2 is a flowchart of a method for generating a feature associated with a signal, by using the Schrödinger operator;

FIG. 3 schematically illustrates as the input data is analyzed using the Schrödinger operator for generating a feature and then classifying the input data based on the feature;

FIG. 4 illustrates MEG signals that are noisy and have multiple peaks;

FIG. 5 illustrates an algorithm that uses the Schrödinger operator for reconstructing a signal;

FIG. 6 illustrates actual results obtained with the novel method discussed herein when applied to a number of patients;

FIG. 7 illustrates the results obtained with the novel method versus the existing methods;

FIG. 8 illustrates the spectrum of the Schrödinger operator for positive and negative eigenvalues;

FIG. 9 is a flowchart of a method for generating a feature based on a signal; and

FIG. 10 is a schematic diagram of a computing device that implements the novel methods discussed herein.

### DETAILED DESCRIPTION

The following description of the embodiments refers to the accompanying drawings. The same reference numbers in different drawings identify the same or similar elements. The following detailed description does not limit the invention. Instead, the scope of the invention is defined by the appended claims. For simplicity, the following embodiments are discussed with regard to MEG signals. However, the methods and systems discussed herein are equally applicable to any signal that exhibits peaks. For example, the methods discussed herein can be applied to water peak estimation, water suppression signal in magnetic resonance spectroscopy (MRS) signals, MRS signal denoising, pulse-shaped signal decomposition and denoising, etc. The novel methods can be integrated in any processing unit to process biomedical signals such as MRS signals, electroencephalogram (EEG) signals, or any other pulse-shaped signal.

Reference throughout the specification to “one embodiment” or “an embodiment” means that a particular feature, structure or characteristic described in connection with an embodiment is included in at least one embodiment of the subject matter disclosed. Thus, the appearance of the phrases “in one embodiment” or “in an embodiment” in various places throughout the specification is not necessarily referring to the same embodiment. Further, the particular features, structures or characteristics may be combined in any suitable manner in one or more embodiments.

According to an embodiment, there is a method that introduces a new characterization of signals. The signals may be spectrum data, biomedical signals or any other type of signal. This new characterization generates new features that can be used for the classification of the signals. The proposed feature generation technique is based on the semi-classical analysis (SCSA) method, which includes the projection of the input signal into a set of functions given by the squared eigenfunctions of the Schrödinger operator associated to the negative eigenvalues, and whose potential is given by the input signal. The computed eigenvalues, eigenfunctions and their different combinations introduce new types of features, which can be used all together or in different combination forms to provide a suitable and accurate discrimination of the data signals.

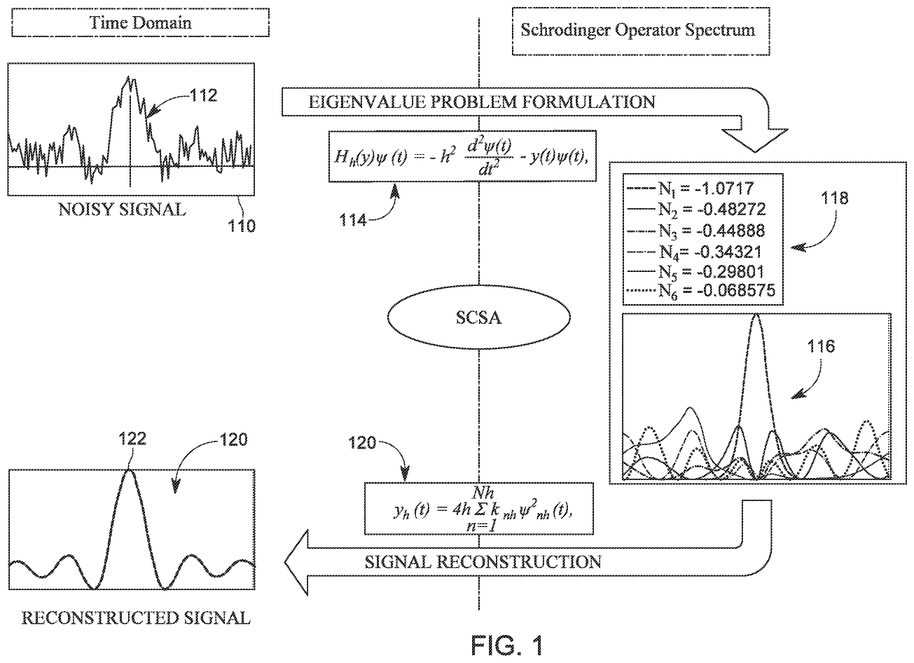

More specifically, as illustrated in FIG. 1, input data 110 is collected in the time domain. The input data 110 includes at least one noisy signal 112. The input data is used with the Schrödinger operator 114 to generate plural eigenfunctions 116, corresponding to plural eigenvalues 118. Then, a feature is generated and this feature is used for selecting significative eigenfunctions. A signal 120 is constructed based on the selected eigenfunctions and the corresponding eigenvalues. The signal 120 has the noise removed and its peak 122 can be easily identified. Having identified the peak of the signal, the classification of the signal can now be performed.

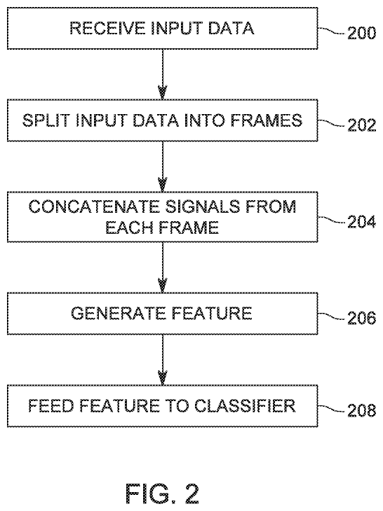

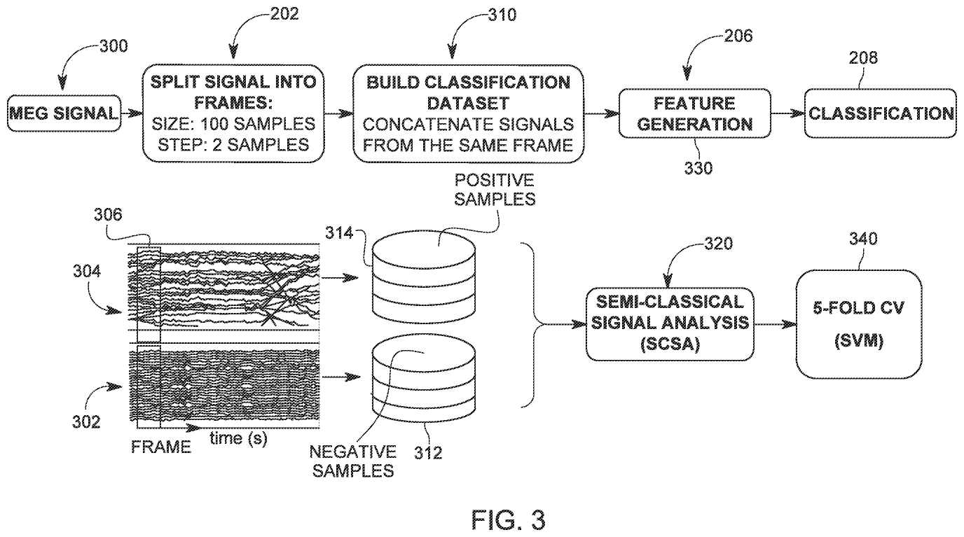

The process illustrated in FIG. 1 is now discussed in more detail. FIG. 2 is a flowchart of a method for generating a feature that characterizes a set of signals (called herein the input data) and uses the generated feature to classify the input data. For a better understanding of the method, actual MEG input data 300 (see FIG. 3) was received in step 200. For this embodiment, the MEG input data has been collected from nine healthy subjects (see data 302) and nine epileptic subjects (see data 304). A total of 18 MEG data segments, each of 15 minutes duration and 26 channels was recorded with a sampling frequency of 1 kHz. The data was then filtered by Spatiotemporal signal space separation method and off-line band-pass filtered for 1-50 Hz. The input data was analyzed by neurologists, which marked the MEG spike locations. The total number of spikes in this input data was found by the neurologists to be about 166.

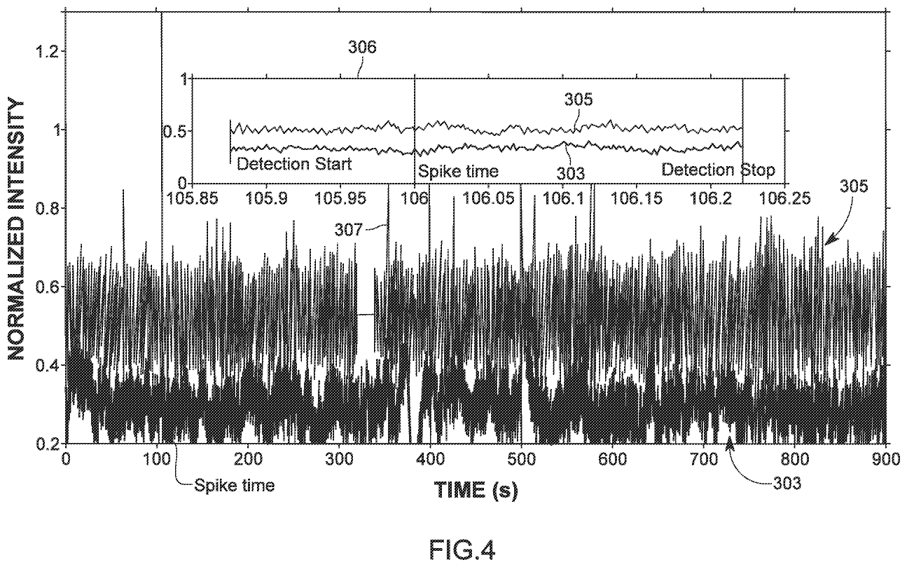

A signal 303 from the input data 302 (i.e., an MEG signal from a healthy subject) and a signal 305 from the input data 304 (i.e., an MEG signal from an epileptic subject) are shown in FIG. 4. The signals are illustrated in this figure as a normalized intensity versus a recording time. The insert of FIG. 4 shows approximately a second worth of the two signals 303 and 305. It is noted that signal 305 exhibits plural spikes 307.

In step 202, the input signal 300 is split into frames, for example, using sliding frames. In one application, a sliding frame 306 includes 100 sample points and the sliding frame slides with a step of 2 samples, i.e., the frame 306 is moved 2 samples and another 100 samples points are considered for a second sliding frame, and so on. The inset of FIG. 4 corresponds to a single sliding frame. Other numbers may be used for the size of the sliding frame and for the step of moving the frame.

The signals from each frame 306 are then concatenated in step 204 to build a classification dataset 310. Note that for this specific embodiment, the MEG signals include 26 channels, i.e., 26 different sensors have been used to collect each MEG signal. For other types of signals, the number of sensors may be fewer or more. Regardless of the number of sensors, the signals in each channel are concatenated for each given frame, to generate a single signal. Two different classes are defined in this embodiment, the negative samples 312 and the positive samples 314. The negative samples 312 include the frames from the healthy subjects 302 and the positive samples 314 include the frames from the epileptic subjects 304. Each class includes the same number of frames.

In step 206, one or more features is generated for these signals. For this step, the semi-classical signal analysis (SCSA) 320 is applied. The SCSA analysis is now discussed in more detail. The SCSA uses signal-dependent functions given by the squared eigenfunctions of the Schrödinger operator to decompose the signal (see, for example, [4] and [5]). The potential V of the Schrödinger operator H(y), in this case, is given by the positive function y(t) representing the signal. The Schrödinger operator is written as follows:

$$H = - {h}^{2} \frac{{d}^{2}}{{dt}^{2}} - V , \qquad ( 1 )$$

where H is the Schrödinger operator, V is the potential, h is a constant, t is the time, and d indicates a derivative. For the SCSA analysis, the potential V is selected to be the positive function y(t) representing the signal, which means that equation (1) becomes:

$$H ( y ) = - {h}^{2} \frac{{d}^{2}}{{dt}^{2}} - y ( t ) . \qquad ( 2 )$$

The Schrödinger equation for the SCSA analysis is given by:

H(y)ψ(t)=λψ(t), (3)

where ψ(t) is the eigenfunction of the Schrödinger operator, and is the eigenvalue of the Schrödinger operator.

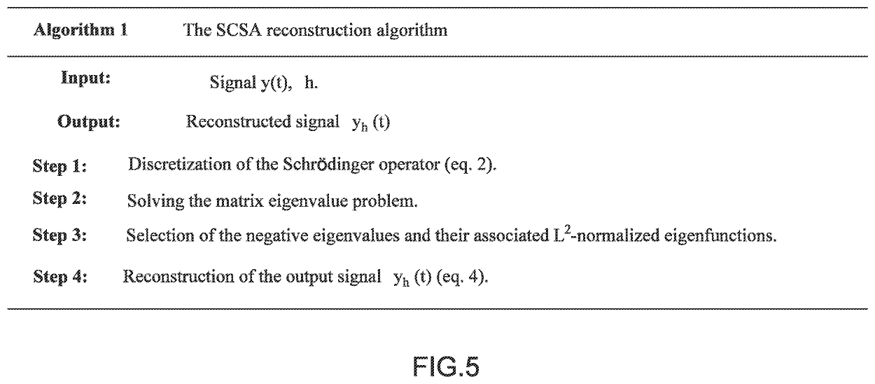

Based on the SCSA analysis, a real positive input signal y(t) can be approximated by plural signals yₕ(t), which are given by:

$${y}_{h} ( t ) = 4 h {\sum }_{n = 1}^{{N}_{h}} \sqrt{- {λ}_{nh}} {ψ}_{nh}^{2} ( t ) , \qquad ( 4 )$$

where n varies from 1 to Nₕ, and n represents the negative eigenvalues λₙₕ of the Schrödinger operator H(y). In addition, λ₁ₕ< . . . <λₙₕ<0. The accuracy of the reconstructed signal yₕ(t) depends on the value of h and also on how many negative eigenvalues λₙₕ are used. Equation (4) provides an exact reconstruction of the original signal y(t) when h converges to zero. When the value of h decreases, the number of eigenvalues λₙₕ increases and the reconstruction improves. However, as the h converges to zero, the calculation amount increases and may become unpractical for a practical application for which the computer power is limited. Thus, for a real situation, a balance between the value of h and the amount of computational power necessary to reconstruct the signal needs to be found. FIG. 5 schematically illustrates the SCSA reconstruction algorithm.

The SCSA analysis is used in step 206 to generate a feature associated with the signals 302 and 304. The SCSA analysis has been used for reconstruction and de-noising of some biomedical signals such as the Magnetic Resonance Spectroscopy (MRS) spectra and the Arterial Blood Pressure (ABP) [6], [7]. Due to the localized and shape-dependent structure of the squared eigenfunctions ψₙₕ² of the Schrödinger operator, the SCSA analysis introduces an effective analysis tool for pulse shaped signals (signals with peaks).

In this embodiment, the feature 330 is related to the number of negative eigenvalues that are used to reconstruct the signal for each frame 306. For this purpose, the parameter Nₕ^{*} is introduced as being the lower feature size and it is defined as:

Nₕ^{*}=min(Nₕ₁,Nₕ₂,N_{hM}), (5)

where Nₕᵢ is the number of negative eigenvalues of the i^{th} frame for a given value h, and M is number of frames. This means that for each frame i, a corresponding number Nₕᵢ of negative eigenvalues λnh is selected in step 206 to reconstruct the signal for that frame, and then, based on equation (5), the minimum number of negative eigenvalues is selected for all the frames. Thus, for each frame of the M frames used in these calculations, only Nₕ^{*} negative eigenvalues are used for reconstructing the signals, and the Nₕ^{*} negative eigenvalues is the generated feature 330.

One way to select the number Nₕᵢ of negative eigenvalues λₙₕ that is used to reconstruct the signal for each frame, is to define a set threshold value. Then, for a given instant, reconstruct the signal with a given number of negative eigenvalues λₙₕ and calculate a different between the original signal and the reconstructed signal at the given instant. If the difference is smaller than the set threshold value, the given number of negative eigenvalues corresponds to Nₕᵢ. If not, increase the given number of negative eigenvalues and evaluate again the difference between the original signal and the reconstructed signal. Repeat this process until the difference is smaller than the set threshold value and that is the value of the given number of negative eigenvalues. This is only one way to determine the Nₕᵢ for each frame. Other criteria may be used for selecting the negative eigenvalues λₙₕ that reproduce the original signal for each frame.

The feature 330 is fed in step 208 to a classifier 340 for classifying the reconstructed signals. The classifier 340 may be, for example, a Support Vector Machine (SVM) predictive model. The SVM model may be developed in 5-fold cross-validation (CV) process with the following subjects: 1734 spiky frames and 1734 healthy frames from different MEG test sessions of the eight healthy and eight epileptic patients.

The performance of the classifier 340 has been measured using the average accuracy, the sensitivity, the specificity and other metrics defined as follows:

$$Accuracy = \frac{1}{5} {\sum }_{n = 1}^{5} \frac{{TP}_{n} + {TN}_{n}}{{TP}_{n} + {FP}_{n} + {TN}_{n} + {FN}_{n}} \times 100 , \\ Sensitivity = \frac{1}{5} {\sum }_{n = 1}^{5} \frac{{TP}_{n}}{{TP}_{n} + {FN}_{n}} \times 100 , \\ Specificity = \frac{1}{5} {\sum }_{n = 1}^{5} \frac{{FP}_{n}}{{FP}_{n} + {TN}_{n}} \times 100 , \\ Precision = \frac{1}{5} {\sum }_{n = 1}^{5} \frac{{TP}_{n}}{{TP}_{n} + {FP}_{n}} \times 100 , \\ {G}_{mean} = \sqrt{Sensitivity \times ( 1 - Specificity )} , and \\ {F}_{1} - Score = \frac{2 \times Precision \times Sensitivity}{Precision + Sensitivity} , \qquad ( 6 )$$

where TPₙ, FPₙ, TNₙ, and FNₙ are the True Positive, False Positive, True Negative, and False Negative values, respectively, for the n^{th} fold. Note that these values are calculated by comparing the results of the classifier 340 made in step 208, and the actual peaks determined by the expert neurologists based on the input data 300.

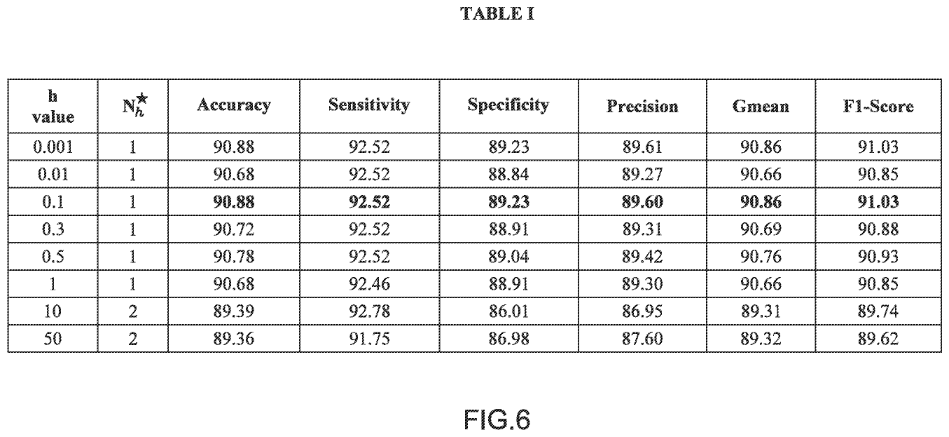

Table 1 (see FIG. 6) shows that with the same average number of negative eigenvalues Nₕ^{*}, lower values of h improves the classification performance. This is an expected result because lower values of h improve the accuracy of the reconstruction as shown in [4]. However, the value of h cannot be very small due to the limited number of points, which limits the number of negative eigenvalues that can be numerically computed.

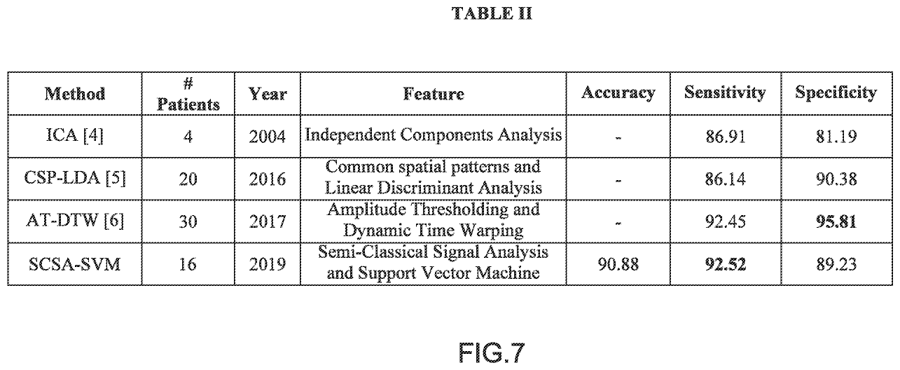

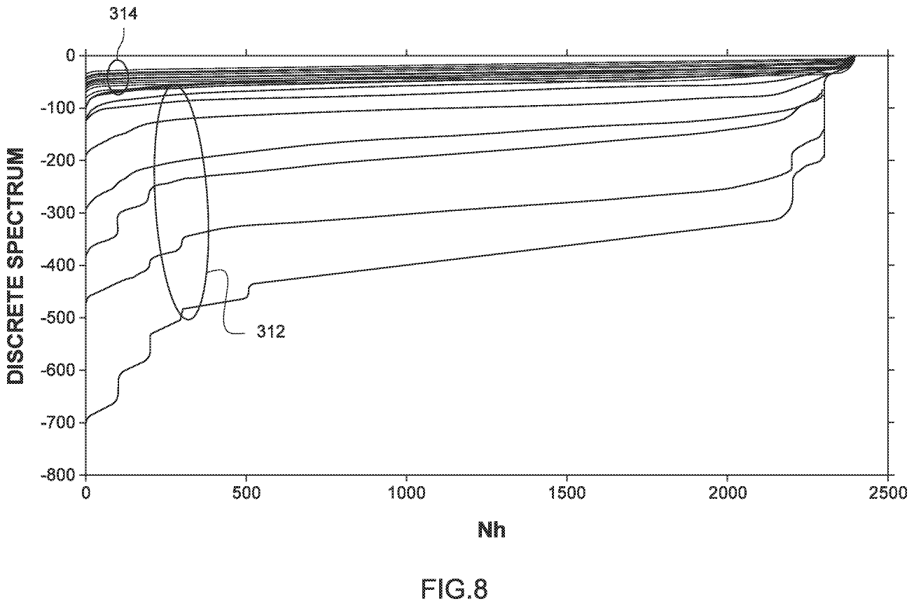

The results of the method illustrated in FIG. 2 were compared to the existing spikes detection approaches using MEG signal reported in [3]. In this regard, Table II (see FIG. 7) shows that the SCSA-based features improve the detection sensitivity and achieves higher specificity compared to two of the existing methods. Moreover, the method of FIG. 2 reduces the generated feature's size to one. This is shown in FIG. 8, which shows that the spectrum (represented on the Y axis of the figure) of the Schrödinger operator separates the positive class 314 from the negative class 312 as only the first Nₕ^{*} negative eigenvalues are used as the generated feature, which are the most dominant and representative eigenvalues. Note that FIG. 8 plots the spectrum of the Schrödinger operator versus the number of negative eigenvalues Nₕ, and for this specific example, Nₕ^{*}=1. Thus, this algorithm reduces the size of the feature to one or a similar small number, which becomes very efficient from a computational point of view

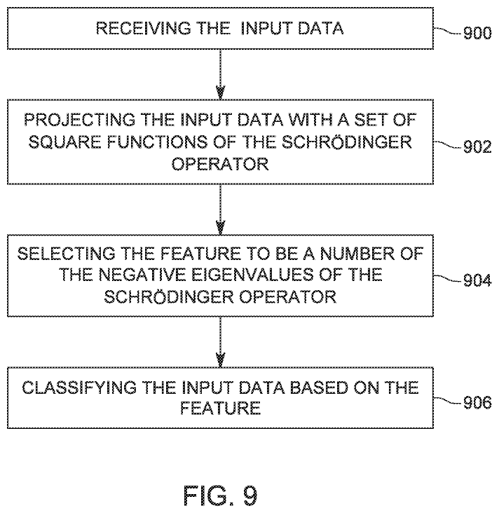

A method for generating a feature having a small size is now discussed with regard to FIG. 9. The includes a step 900 of receiving the input data, a step 902 of projecting the input data with a set of square functions ψₙₕ² of the Schrödinger operator, a step 904 of selecting the feature to be a number of the negative eigenvalues λₙₕ of the Schrödinger operator, and a step 906 of classifying the input data based on the feature. The method may also include a step of splitting the input data into frames, and/or concatenating plural signals from a frame to form a single signal, and/or using the single signal as a potential for the Schrödinger operator, and/or reconstructing the single signal using the set of square functions λₙₕ² of the Schrödinger operator and the number of the negative eigenvalues λₙₕ of the Schrödinger operator, and/or identifying a peak of the reconstructed single signal. In one application, the step of classifying includes classifying the input data based on the peak.

The methods discussed herein advantageously present a new feature generation and dimensionality reduction algorithm. While the algorithm has been presented as a specific application for epileptic spikes detection in MEG signals, the same algorithm may be used for any signal that requires spike identification. The algorithm projects an input signal into the discrete spectrum of the Schrödinger operator and then selects a number of eigenvalues to be used for regenerating the signal from the eigenfunctions of the Schrödinger operator. As illustrated in FIGS. 6 and 7, the novel algorithm obtains the highest sensitivity up to 92.52% with a specificity of 89.10% for a given dataset.

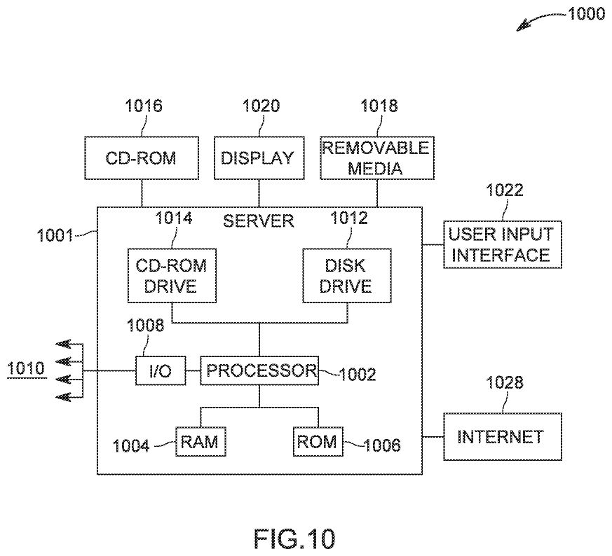

The above-discussed procedures and methods may be implemented in a computing device as illustrated in FIG. 10. Hardware, firmware, software or a combination thereof may be used to perform the various steps and operations described herein.

Computing device 1000 suitable for performing the activities described in the embodiments discussed above may include a server 1001. Such a server 1001 may include a central processor (CPU) 1002 coupled to a random access memory (RAM) 1004 and to a read-only memory (ROM) 1006. ROM 1006 may also be other types of storage media to store programs, such as programmable ROM (PROM), erasable PROM (EPROM), etc. Processor 1002 may communicate with other internal and external components through input/output (I/O) circuitry 1008 and bussing 1010 to provide control signals and the like. Processor 1002 carries out a variety of functions as are known in the art, as dictated by software and/or firmware instructions.

Server 1001 may also include one or more data storage devices, including hard drives 1012, CD-ROM drives 1014 and other hardware capable of reading and/or storing information, such as DVD, etc. In one embodiment, software for carrying out the above-discussed steps may be stored and distributed on a CD-ROM or DVD 1016, a USB storage device 1018 or other form of media capable of portably storing information. These storage media may be inserted into, and read by, devices such as CD-ROM drive 1014, disk drive 1012, etc. Server 1001 may be coupled to a display 1020, which may be any type of known display or presentation screen, such as LCD, plasma display, cathode ray tube (CRT), etc. A user input interface 1022 is provided, including one or more user interface mechanisms such as a mouse, keyboard, microphone, touchpad, touch screen, voice-recognition system, etc.

Server 1001 may be coupled to other devices, such as medical instruments, detectors, sensors, etc. The server may be part of a larger network configuration as in a global area network (GAN) such as the Internet 1028, which allows ultimate connection to various landline and/or mobile computing devices.

The disclosed embodiments provide a method and system that is capable to detect and classify peaks associated with one or more signals. The embodiments are intended to cover alternatives, modifications and equivalents, which are included in the spirit and scope of the invention as defined by the appended claims. Further, in the detailed description of the embodiments, numerous specific details are set forth in order to provide a comprehensive understanding of the claimed invention. However, one skilled in the art would understand that various embodiments may be practiced without such specific details.

Although the features and elements of the present embodiments are described in the embodiments in particular combinations, each feature or element can be used alone without the other features and elements of the embodiments or in various combinations with or without other features and elements disclosed herein.

This written description uses examples of the subject matter disclosed to enable any person skilled in the art to practice the same, including making and using any devices or systems and performing any incorporated methods. The patentable scope of the subject matter is defined by the claims, and may include other examples that occur to those skilled in the art. Such other examples are intended to be within the scope of the claims.

### REFERENCES

[1] A. Ossadtchi, S. Baillet, J. C. Mosher, D. Thyerlei, W. Sutherling, and R. M. Leahy, “Automated interictal spike detection and source localization in magnetoencephalography using independent components analysis and spatiotemporal clustering,” Clinical Neurophysiology, vol. 115, no. 3, pp. 508-522, 2004.
[2] M. I. Khalid, T. Alotaiby, S. A. Aldosari, S. A. Alshebeili, M. H. Al-Hameed, F. S. Y. Almohammed, and T. S. Alotaibi, “Epileptic MEG Spikes Detection Using Common Spatial Patterns and Linear Discriminant Analysis,” IEEE Access, vol. 4, pp. 4629-4634, 2016.
[3] M. I. Khalid, T. N. Alotaiby, S. A. Aldosari, S. A. Alshebeili, M. H. Alhameed, and V. Poghosyan, “Epileptic MEG Spikes Detection Using Amplitude Thresholding and Dynamic Time Warping,” IEEE Access, vol. 5, pp. 11 658-11 667, 2017.
[4] T.-M. Laleg-Kirati, E. Crepeau, and M. Sorine, “Semi-classical signal analysis,” Mathematics of Control, Signals, and Systems, vol. 25, no. 1, pp. 37-61, 2013.
[5] B. Helffer and T.-M. Laleg-Kirati, “On semi-classical questions related to signal analysis,” Asymptotic Analysis.
[6] T.-M. Laleg, E. Crepeau, Y. Papelier, and M. Sorine, “Arterial blood pressure analysis based on scattering transform i,” in Engineering in Medicine and Biology Society, 2007. EMBS 2007. 29th Annual International Conference of the IEEE. IEEE, 2007, pp. 5326-5329.
[7] T. Laleg-Kirati, J. Zhang, E. Achten, and H. Serrai, “Spectral data de-noising using semi-classical signal analysis: application to localized MRS,” NMR in Biomedicine, vol. 29, no. 10, pp. 1477-1485, 2016.

1. A method for generating a feature associated with input data, the method comprising:
receiving the input data;
projecting the input data with a set of square functions ψₙₕ² of the Schrödinger operator;
selecting the feature to be a number of the negative eigenvalues λₙₕ of the Schrödinger operator; and
classifying the input data based on the feature.

2. The method of claim 1, wherein the set of square functions ψₙₕ² is associated with the negative eigenvalues λₙₕ of the Schrödinger operator.

3. The method of claim 1, further comprising:
splitting the input data into frames.

4. The method of claim 3, further comprising:
concatenating plural signals from a frame to form a single signal.

5. The method of claim 4, further comprising:
using the single signal as a potential for the Schrödinger operator.

6. The method of claim 5, further comprising:
reconstructing the single signal using the set of square functions ψₙₕ² of the Schrödinger operator and the number of the negative eigenvalues λₙₕ of the Schrödinger operator.

7. The method of claim 6, further comprising:
identifying a peak of the reconstructed single signal.

8. The method of claim 7, wherein the step of classifying comprises:
classifying the input data based on the peak.

9. The method of claim 1, wherein the input data is a magnetoencephalography signal.

10. The method of claim 9, wherein the step of classifying comprises:
identifying a signal from the input data that indicates an epileptic patient.

11. The method of claim 1, wherein the feature is a minimum number of negative eigenvalues for each of the frames.

12. A computing device for generating a feature associated with input data, the computing device comprising:
an interface for receiving the input data; and
a processor connected to the interface and configured to,
project the input data with a set of square functions ψₙₕ² of the Schrödinger operator;
select the feature to be a number of the negative eigenvalues λₙₕ of the Schrödinger operator; and
classify the input data based on the feature.

13. The computing device of claim 12, wherein the set of square functions ψₙₕ² is associated with the negative eigenvalues λₙₕ of the Schrödinger operator.

14. The computing device of claim 12, wherein the processor is further configured to:
split the input data into frames; and
concatenate plural signals from a frame to form a single signal.

15. The computing device of claim 14, wherein the processor is further configured to:
use the single signal as a potential for the Schrödinger operator; and
reconstruct the single signal using the set of square functions ψₙₕ² of the Schrödinger operator and the number of the negative eigenvalues λₙₕ of the Schrödinger operator.

16. The computing device of claim 15, wherein the processor is further configured to:
identify a peak of the reconstructed single signal; and
classify the input data based on the peak.

17. The computing device of claim 12, wherein the input data is a magnetoencephalography signal and wherein the processor is further configured to identify a signal from the input data that indicates an epileptic patient.

18. A non-transitory computer readable medium including computer executable instructions, wherein the instructions, when executed by a processor, implement instructions for generating a feature associated with input data, the instructions comprising:
receiving the input data;
projecting the input data with a set of square functions ψₙₕ² of the Schrödinger operator;
selecting the feature to be a number of the negative eigenvalues λₙₕ of the Schrödinger operator; and
classifying the input data based on the feature.

19. The medium of claim 18, wherein the set of square functions ψₙₕ² is associated with the negative eigenvalues λₙₕ of the Schrödinger operator.

20. The medium of claim 18, further comprising:
splitting the input data into frames;
concatenating plural signals from a frame to form a single signal;
using the single signal as a potential for the Schrödinger operator;
reconstructing the single signal using the set of square functions ψₙₕ² of the Schrödinger operator and the number of the negative eigenvalues λₙₕ of the Schrödinger operator;
identifying a peak of the reconstructed single signal; and
classifying the input data based on the peak.
